SEQUENCE LISTING (26 sequences)

SEQ ID NO: 1 - DNA, 22 bp, Unknown
  The linker strands, linker primers, and universal primers shown herein are universal bacterial species markers for use in identifying any bacterial species according to certain embodiments of the present invention.
ctaaggccttgctagcagaagc

SEQ ID NO: 2 - DNA, 26 bp, Unknown
  The linker strands, linker primers, and universal primers shown herein are universal bacterial species markers for use in identifying any bacterial species according to certain embodiments of the present invention.
gcttctgctagcaaggccttagaaaa

SEQ ID NO: 3 - DNA, 20 bp, Unknown
  The linker strands, linker primers, and universal primers shown herein are universal bacterial species markers for use in identifying any bacterial species according to certain embodiments of the present invention.
taaggccttgctagcagaag

SEQ ID NO: 4 - DNA, 20 bp, Unknown
  The linker strands, linker primers, and universal primers shown herein are universal bacterial species markers for use in identifying any bacterial species according to certain embodiments of the present invention.
agagtttgatcctggctcag

SEQ ID NO: 5 - DNA, 20 bp, Unknown
  The linker strands, linker primers, and universal primers shown herein are universal bacterial species markers for use in identifying any bacterial species according to certain embodiments of the present invention.
ctgagccaggatcaaactct

SEQ ID NO: 6 - DNA, 18 bp, Unknown
  The linker strands, linker primers, and universal primers shown herein are universal bacterial species markers for use in identifying any bacterial species according to certain embodiments of the present invention.
tcctacgggaggcagcag

SEQ ID NO: 7 - DNA, 21 bp, Unknown
  The linker strands, linker primers, and universal primers shown herein are universal bacterial species markers for use in identifying any bacterial species according to certain embodiments of the present invention.
agataccctggtagtccacgc

SEQ ID NO: 8 - DNA, 19 bp, Unknown
  The linker strands, linker primers, and universal primers shown herein are universal bacterial species markers for use in identifying any bacterial species according to certain embodiments of the present invention.
cggtgaatacgttcccggg

SEQ ID NO: 9 - DNA, 18 bp, Unknown
  The linker strands, linker primers, and universal primers shown herein are universal bacterial species markers for use in identifying any bacterial species according to certain embodiments of the present invention.
gtacacaccgcccgtcac

SEQ ID NO: 10 - DNA, 19 bp, Unknown
  The linker strands, linker primers, and universal primers shown herein are universal bacterial species markers for use in identifying any bacterial species according to certain embodiments of the present invention.
cagtaccgtgagggaaagg

SEQ ID NO: 11 - DNA, 20 bp, Unknown
  The linker strands, linker primers, and universal primers shown herein are universal bacterial species markers for use in identifying any bacterial species according to certain embodiments of the present invention.
tcgcctttccctcacggtac

SEQ ID NO: 12 - DNA, 21 bp, Unknown
  The linker strands, linker primers, and universal primers shown herein are universal bacterial species markers for use in identifying any bacterial species according to certain embodiments of the present invention.
gatggctgcttctaagccaac

SEQ ID NO: 13 - DNA, 21 bp, Unknown
  The linker strands, linker primers, and universal primers shown herein are universal bacterial species markers for use in identifying any bacterial species according to certain embodiments of the present invention.
ggaggttggcttagaagcagc

SEQ ID NO: 14 - DNA, 22 bp, Unknown
  The linker strands, linker primers, and universal primers shown herein are universal bacterial species markers for use in identifying any bacterial species according to certain embodiments of the present invention.
cgtgaggacagaactgaacagg

SEQ ID NO: 15 - DNA, 21 bp, Unknown
  The linker strands, linker primers, and universal primers shown herein are universal bacterial species markers for use in identifying any bacterial species according to certain embodiments of the present invention.
ttctgtcctcacggccaaagg

SEQ ID NO: 16 - DNA, 19 bp, Unknown
  The linker strands, linker primers, and universal primers shown herein are universal bacterial species markers for use in identifying any bacterial species according to certain embodiments of the present invention.
ccggtcctctcgtactagg

SEQ ID NO: 17 - DNA, 23 bp, Unknown
  The linker strands, linker primers, and universal primers shown herein are universal bacterial species markers for use in identifying any bacterial species according to certain embodiments of the present invention.
cggtcctaaggtagcgaaattcc

SEQ ID NO: 18 - DNA, 23 bp, Unknown
  The linker strands, linker primers, and universal primers shown herein are universal bacterial species markers for use in identifying any bacterial species according to certain embodiments of the present invention.
ggaatttcgctaccttaggaccg

SEQ ID NO: 19 - DNA, 20 bp, Unknown
  The linker strands, linker primers, and universal primers shown herein are universal bacterial species markers for use in identifying any bacterial species according to certain embodiments of the present invention.
cttgtcgggtaagttccgac

SEQ ID NO: 20 - DNA, 20 bp, Unknown
  The linker strands, linker primers, and universal primers shown herein are universal bacterial species markers for use in identifying any bacterial species according to certain embodiments of the present invention.
gtcggaacttacccgacaag

SEQ ID NO: 21 - DNA, 20 bp, Unknown
  The linker strands, linker primers, and universal primers shown herein are universal bacterial species markers for use in identifying any bacterial species according to certain embodiments of the present invention.
gagacagttcggtccctatc

SEQ ID NO: 22 - DNA, 20 bp, Unknown
  The linker strands, linker primers, and universal primers shown herein are universal bacterial species markers for use in identifying any bacterial species according to certain embodiments of the present invention.
gatagggaccgaactgtctc

SEQ ID NO: 23 - DNA, 20 bp, Unknown
  The linker strands, linker primers, and universal primers shown herein are universal bacterial species markers for use in identifying any bacterial species according to certain embodiments of the present invention.
cccatgccgaacacagaagt

SEQ ID NO: 24 - DNA, 20 bp, Unknown
  The linker strands, linker primers, and universal primers shown herein are universal bacterial species markers for use in identifying any bacterial species according to certain embodiments of the present invention.
acttctgtgttcggcatggg

SEQ ID NO: 25 - DNA, 18 bp, Unknown
  The linker strands, linker primers, and universal primers shown herein are universal bacterial species markers for use in identifying any bacterial species according to certain embodiments of the present invention.
gaggaaagtccgggctcc

SEQ ID NO: 26 - DNA, 18 bp, Unknown
  The linker strands, linker primers, and universal primers shown herein are universal bacterial species markers for use in identifying any bacterial species according to certain embodiments of the present invention.
ggtgcggtaagagcgcac

(19) United States
(12) Patent Application Publication
(10) Pub. No.: US 2009/0253121 A1
(43) Pub. Date: Oct. 8, 2009

(54) METHOD FOR AMT-RFLP DNA FINGERPRINTING

(76) Inventor: Micah Halpern, Liberty, MO (US)

Correspondence Address: HUSCH BLACKWELL SANDERS LLP, 4801 Main Street, Suite 1000, KANSAS CITY, MO, 64112, US

(21) Appl. No.: 12/062,832

(22) Filed: Apr. 4, 2008

(51) Int. Cl. C12Q 1/70; C12Q 1/68

(52) U.S. Cl. 435/5; 435/6

(57) ABSTRACT

An improved method for identifying and classifying organisms is provided. The method comprises the steps of obtaining a sample of DNA, isolating a portion of the sample to be analyzed, cutting the sample portion into at least two fragments, ligating both ends of the fragments with double-stranded linker sequences, adding the fragments to a polymerase chain reaction that amplifies a diagnostic region using a SYBR green-intercalating dye, and analyzing the fragments using a melt-curve analysis.

### BACKGROUND OF THE INVENTION

The identification and classification of organisms, including bacteria, is an important and on-going process. Known methods for determining the identification and classification of organisms include biochemical and molecular techniques. Molecular methods for identification and classification are generally accepted as more robust techniques than biochemical methods. Molecular techniques include, but are not limited to, Southern blot probing, standard/real-time polymerase chain reaction (PCR) and repetitive element PCR. Each of these techniques has limited throughput and/or a limited ability to discriminate organisms. Southern blot probing requires large quantities of deoxyribonucleic acid (DNA), specialty reagents, specialty treatment, and trained personnel. Standard PCR techniques target one or few specific organisms. In addition, standard PCR requires follow-on analysis using gels, sequencing, or restriction fragment length polymorphism (RFLP) for bacterial identification. This can be time-consuming and requires specialty equipment.

Real-time PCR has similar limitations to standard PCR but does not require follow-on analysis for identification. Rather, identification is accomplished simultaneously using a labeled probe. Standard or real-time PCR cannot be used to look for an unspecified species of bacteria in an unknown sample. Repetitive element PCR requires upfront characterization of a sample which necessitates growth of the organism. No confirmation is possible due to the complex pattern that is generated by this technique because the loci generated are non-specific and the kits currently available are not universal for all bacteria. Therefore, it would be beneficial to provide a method of identification of organisms that has a high throughput and the ability to effectively discriminate organisms. It would also be beneficial to provide a method of identification that needs a minimum amount of skilled labor and specialty equipment.

Biochemical identification is more labor intensive than molecular methods and does not always provide a discrete answer. Biochemical techniques include staining, differential media and identification by substrate usage. Additionally, biochemical techniques require growth of the organism, specialty reagents, specialty equipment and highly trained personal. Therefore, it would be beneficial to provide a method of identification that is inexpensive, utilized by minimally-skilled personnel and does not use special equipment.

### SUMMARY OF THE INVENTION

In one of many illustrative, non-limiting aspects of the present invention, there is provided a method for identifying and classifying organisms. The method includes obtaining a sample of DNA, isolating a portion of the sample to be analyzed, cutting the sample portion into at least two fragments, ligating both ends of the fragments with double-stranded linker sequences, adding the fragments to a polymerase chain reaction that amplifies a diagnostic genetic region using a SYBR green-intercalating dye, and analyzing the fragments using a melt-curve analysis.

### DETAILED DESCRIPTION OF THE INVENTION

There is provided herein a method for the identification and classification of organisms using amplified melt terminal restriction fragment length polymorphism (AMT-RFLP) DNA fingerprinting. The method of the present invention is used for fingerprinting bacteria or other organisms for species identification. The present method may be used on any source of DNA such as fungal, plant, bacterial, animal or combinations thereof. The present method can be used on both dead and live cultured organisms.

For purposes of illustration, the method of the present invention is described as if the sample being analyzed is bacterial in nature. It will be appreciated by one skilled in the art that the method of the present invention may also be used to analyze any source of DNA by interrogating genetic regions commonly targeted for species level identification in the organism of interest.

As an illustrative example, first, a clean sample of DNA is obtained by extraction. Extraction techniques now known or hereafter developed may be used to extract a sample of DNA to be analyzed. As an illustrative example, from about 0.1 nanograms to about 1000 nanograms of DNA is extracted. The DNA sample is then enzymatically cut by a restriction endonuclease that recognizes a specific DNA cut site. The cut sample is next treated with a phosphatase that removes phosphates from the ends of every fragment. This step prevents ligation of fragments together in the following step wherein double-stranded linker sequences are ligated to both ends of every fragment in the sample. Double-stranded linkers are composed of known non-specific DNA sequences. The sample treatment steps described hereinabove are a known standard technique used for different versions of ligation-mediated PCR. It will be appreciated that other sample treatment methods now known or hereafter discovered may also be used in the method of the present invention.

A portion of the treated sample is then added as a template to a PCR using a dye and, preferably, an intercalating dye. As used herein, an intercalating dye is any dye that is capable of being inserted between bases of nucleic acids. Preferred intercalating dyes fluoresce upon incorporation within double-stranded DNA. When the double-stranded DNA is denatured, the intercalating dye is released and ceases to fluoresce. As an illustrative example, the following dyes are suitable for use in the present invention: SYBR green-intercalating dyes, cyanine dimer dyes, ethidium bromide dyes and acridine dyes may be used. One primer complementary to the ligated linker sequence and one primer complementary to the universal DNA sequence are used in the amplification. The universal DNA priming site consists of approximately 20 nucleotides of sequence that is conserved among all bacteria.

The amplification step creates multiple copies of a region of DNA that is diagnostic of different bacteria. Differential fragment sizes and/or sequences generated from this technique are diagnostic for different bacteria. SYBR green in the reaction is used for a follow-on melt-curve analysis. This is achieved by denaturing both strands of amplified DNA at a temperature of approximately 95° C. The temperature is then lowered to a point where both strands of DNA reassemble and SYBR green dye is deposited between the strands. As a result of intercalation, the dye fluoresces at a particular wavelength that is detected by filters in the real-time thermalcycler. Readings are taken as the temperature is slowly increased. As the temperature reaches a critical point where the two strands of the fragment begin to break apart, SYBR green dye is released into solution where it no longer fluoresces. This decreases the signal and a melt point is achieved when 50% of the product denatures.

Melt point temperatures are dependent on both product size and sequence. Larger products melt at higher temperatures than smaller products and GC-rich templates melt at higher temperatures than AT-rich templates. The amplified product for each different bacterium differs either in size (dependent on restriction site recognition) and/or sequence composition that is specific to genus and species designations. Hence, a different melt curve is produced depending on the bacteria being examined. The combination of the ligation-mediated PCR and the follow-on melt-curve analysis produces a rapid identification of the unknown sample.

No one genetic marker is able to produce a unique melt curve for all bacteria and, more specifically, closely-related species. This necessitates the combination of four universal loci that together produce a unique DNA fingerprint pattern for each species of bacteria. Patterns are compared against a database of known species to aid in the identification of unknown samples. This same approach can be applied for species identification of fungal, plant and/or animal species by simply replacing primers in the PCR step with appropriate genetic markers for the organism of interest.

The present invention is further illustrated by the following examples that are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLES

In the following examples, the methods of the present invention were evaluated and optimized for four different loci within the 16S-23S rRNA operon wherein the methods of the present invention were able to distinguish among 35 different species. Locus-specific primers were combined with linker-specific primers to generate PCR amplicons that were discriminated by melt-curve analysis in a PCR master mix containing SYBR Green intercalating dye. Because of the increased number of hydrogen bonds, longer amplicons and GC-rich sequences denatured at higher temperatures than shorter amplicons and AT-rich sequences. It was found that melt-curve analysis often surpassed size separation (e.g., electrophoresis) because it discriminates among equal length fragments of differing nucleotide content (e.g., sequence). As shown in the following Table 1, three samples were separated by size using electrophoresis as well as melt curve profiles. Melt curve analysis provided unique identifiers for the three samples whereas size separation did not.

TABLE 1

| Genus species | Size (bp) | Melt (degree) |
|---|---|---|
| Streptococcus pneumoniae | 159 | 86.0 |
| Pseudomonas aeruginosa | 159 | 87.0 |
| Pantoea ananatis | 159 | 88.4 |

Using the methods of the present invention, individual species were found to render a unique melt profile that is defined both by size (due to differences in downstream restriction enzyme recognition sequences) and amplicon sequence across included loci. The resulting pattern, which provides a unique fingerprint for each species, derived from a combination of differences in a downstream restriction site and/or sequence differences in the included (amplified) bacterial DNA. Preferred restriction enzymes include, but are not limited to, AluI, Ca c81, NIa IV, HpyCH4V, BstU I, Rsa I, HaeIII, and combinations thereof. Preferred loci include, but are not limited to, 16S rRNA, 23S rRNA, 5S rRNA, Rnase P, and combinations thereof.

DNA extracts were subjected to LM-PCR sample processing. All reagents were supplied by commercial sources. Briefly, extracted DNA samples were quantitated using an intercalating dye (Picogreen—Molecular Probes) and a standard plate reader. Quantitated samples were adjusted to 1 ng/μL concentrations using molecular grade water. 20 ng were incubated with a restriction enzyme cocktail utilizing 20 units of Alu I (New England Biolabs), a blunt cutter that recognizes ‘AGCT’. One unit of enzyme is the amount required to digest 1 ug of DNA in 1 hour at 37 degrees. Hence, 1000 fold more enzyme was used to insure complete digestion. Under conditions of high enzyme concentration, some restriction enzymes can cleave a substrate outside the recognition sequence (star activity). The primary restriction endonuclease used (Alu I) does not exhibit this property. If ample discrimination is not achieved, another blunt four base cutter lacking star activity (BstU I, Cac8 I, Hae III, HpyCH4V, Nla IV and Rsa I) may be utilized. The current blunt linker sequence is compatible with all enzymes listed.

Compatible reagents and enzyme for subsequent dephosphorylation (New England Biolabs) were added directly to the restriction digestion. The Antarctic Phosphatase (NEB #M0289) catalyzes the removal of 5′ phosphate groups from DNA. Since phosphatase-treated fragments lack the 5′ phosphoryl termini required by ligases, they cannot self-ligate. Antarctic Phosphatase (NEB #M0289) is completely heat-inactivated in 5 minutes at 65° C. avoiding the concern of downstream complications and allows one to proceed directly to the ligation reaction without further purification of DNA. It is important to inactivate the phosphatase because residual active phosphatase will cause failure in subsequent ligation reactions.

Reagents and enzyme required for ligation were added directly to the dephosphorylation. Double-stranded blunt linkers used in the reaction consist of complementary oligonucleotide sequences that lack cross-reactivity with any publicly available microbial sequence. One linker strand is synthesized with a 5′ phosphate that catalyzes ligation to 5′ phosphate deficient fragments and a 3′ AAAA tail to prevent linker concatemers from forming. No more than two linkers have the ability to ligate together due to the poly AAAA tail. This significantly reduces background during subsequent PCR amplification. The ligation reaction reaches completion in 5 minutes at room temperature due to Polyethylene Glycol (PEG) and high concentration (2000 Units/μL) ligase included in the reaction (NEB #M2200L).

An aliquot of the prepared sample was subjected to PCR amplification for species markers. In an effort to further reduce linker dimer background, processed samples were purified using commercial 96 well silica plates (Qiagen). Plates contain silica-gel-membranes for binding of DNA in high-salt buffer and elution with water. The purification procedure removes linker dimers, enzymes, salts, and other impurities incorporated into the sample during enzymatic processing. The column retains fragments >100 bp (enzymatically processed DNA) and selectively remove fragments <40 bp (linker dimers). Specialized binding buffers promote selective adsorption of DNA molecules within particular size ranges (100 bp-10 kb) suitable for this protocol (100-1000 bp). Qiagen kits are routinely used in laboratory settings for this type of application. Although adequate results have been obtained without subsequent purification, the resulting increase in signal to noise was preferable during development.

Preferred universal bacterial species markers consist of four conserved priming sites (˜20-25 bp); 1 in 16S rRNA and 3 in 23S rRNA. Any bacterial species can be identified utilizing these four markers in combination with LM-PCR prepared samples and SYBR Green Melt Curve detection. As shown in the following Table 2, however, other primers and linker sequences have been and may be used in accordance with the methods of the present invention.

TABLE 2
LINKER SEQUENCES

| | |
|---|---|
| Linker F strand | CTAAGGCCTTGCTAGCAGAAGC |
| Linker R strand | Phos-GCTTCTGCTAGCAAGGCCTTAGAAAA |
| Linker Primer | TAAGGCCTTGCTAGCAGAAG |

OPTIMAL UNIVERSAL AMT-RFLP PRIMERS

| | |
|---|---|
| 23S-10F | CAGTACCGTGAGGGAAAGG |
| 23S-10R.2 | TCGCCTTTCCCTCACGGTAC |
| 23S-4F | GAGACAGTTCGGTCCCTATC |
| 16S-101 | TCCTACGGGAGGCAGCAG |

UNIVERSAL AMT-RFLP PRIMERS

| | |
|---|---|
| 16S.8F | AGAGTTTGATCCTGGCTCAG |
| 16S-8R | CTGAGCCAGGATCAAACTCT |
| 16S-101 | TCCTACGGGAGGCAGCAG |
| 16S-102 | AGATACCCTGGTAGTCCACGC |
| 16S-103 | CGGTGAATACGTTCCCGGG |
| 16S-104 | GTACACACCGCCCGTCAC |
| 23S-10F | CAGTACCGTGAGGGAAAGG |
| 235-10R.2 | TCGCCTTTCCCTCACGGTAC |
| 23S-21R | GATGGCTGCTTCTAAGCCAAC |
| 235-21F | GGAGGTTGGCTTAGAAGCAGC |
| 23S-22F | CGTGAGGACAGAACTGAACAGG |
| 23S-22R | TTCTGTCCTCACGGCCAAAGG |
| 23S-23R | CCGGTCCTCTCGTACTAGG |
| 23S-24F | CGGTCCTAAGGTAGCGAAATTCC |
| 23S-24R | GGAATTTCGCTACCTTAGGACCG |
| 23S-2F | CTTGTCGGGTAAGTTCCGAC |
| 23S-2R | GTCGGAACTTACCCGACAAG |
| 23S-4F | GAGACAGTTCGGTCCCTATC |
| 23S-4R | GATAGGGACCGAACTGTCTC |
| 5S-F | CCCATGCCGAACACAGAAGT |
| 5S-R | ACTTCTGTGTTCGGCATGGG |
| RnaseP 1 | GAGGAAAGTCCGGGCTCC |
| RnaseP 2 | GGTGCGGTAAGAGCGCAC |

Each amplification reaction utilized an optimized species locus primer, a linker primer and a commercially available PCR mastermix (BioRad iQ SYBR Green Supermix). The effectiveness of BioRad's platforms and reagents have been validated in previous development work. Utilizing a commercial mastermix allows stringent quality control and reproducibility. This same protocol was used for all four species markers. Species markers were amplified using a standard three-step protocol. An elongation step (72 degrees for 1 min) has been included for amplification of larger (>1000 bp) fragments not feasible with a two-step protocol. Similar melting temperatures of all four species identification primers permit an identical annealing step (64 degrees) for all four reactions. Samples are amplified for 33 cycles and increasing signal from intercalated SYBR Green is monitored using BioRad's standard optical module (includes light source, filters and CCD camera). It has been shown in previous work that amplification beyond 33 cycles results in non-specific background amplification. 40 picograms of DNA have consistently produced a species identification signal utilizing this protocol.

Follow-on melt curve analysis was accomplished by denaturing, reannealing and slowly (0.2° C./15 sec) increasing temperature (standard melt curve analysis). This step is appended to the end of the amplification protocol and requires no manipulation of samples. Commercial thermal cyclers utilizing a Peltier heating/cooling block are limited to a ramp rate of 0.2° C. Fluorescence is monitored by a CCD camera located in the optical module as temperature is increased. A drop in signal is indicated at the point where the fragment denatures based on size and GC content due to the release of SYBR Green dye. The negative first derivative is calculated and the melt curve of the fragment is indicated by BioRad's iQ software.

### Example 1

In the following Table 3, summaries of 35 different DNA stocks (ATCC) are provided according to the melt temperature (° C.) determined at each locus. Reported values represent the average of three replicates with a standard deviation ≦+/−0.2° C. between replicates reflecting the limit of temperature control of the Peltier temperature block used in commercial thermal cyclers. With the exception of the closely-related enterobacterium Escherichia coli and Shigella flexneri, all tested species reported unique (four loci) melt peak profiles. Negative controls used included water, herring sperm DNA (HS), and Human cell line K562. In Table 1, a lack of amplification is denoted by the symbol “-” and those species having two melt peaks represent divergence between 16S-23S operon copies.

TABLE 3

| | Locus 1 | Locus 2 | Locus 3 | Locus 4 |
|---|---|---|---|---|
| | 23S A | 23S B | 23S C | 16S A |
| ID | Average | Average | Average | Average |
| Actinomyces naeslundi | — | 88.2 | 89.0 | 88.5 |
| Bacillus cereus | 85.0, 87.5 | 86.0 | 82.2 | 84.7, 87.4 |
| Bacillus globigii | 87.4 | 86.4 | 83.7 | 85.3 |
| Bacillus subtilis | 87.5 | 86.6 | 83.5, 87.1 | 85.1 |
| Bacillus thuringiensis | 85.7, 87.6 | 86.0 | 82.1, 86.1 | 84.8, 87.6 |
| Bacteroides fragilis | — | — | 85.9 | 85.7 |
| Bacteroides vulgatus | 83.6 | 85.8 | 86.2 | 85.5 |
| Bordetella pertussis | 84.5 | 85.8 | 86.6 | 87.5 |
| Borrelia burgdorferi | 84.7 | 82.2, 79.8 | 81.4 | — |
| Campylobacter jejuni | 83.0, 86.0 | 84.6 | 83.7 | 87.0 |
| Clostridium difficile | 82.4 | 84.0 | 85.8 | 87.3 |
| Desulfovibrio vulgaris | 81.9 | 86.6 | 86.7 | 89.3 |
| Enterobacter cloacae | 85.9 | 85.6 | 87.3 | 88.4 |
| Enterococcus faecalis | 82.9 | 85.6 | 83.0 | 88.0 |
| Escherichia coli | 86.1 | 84.8 | 87.8 | 87.4 |
| (EDL933) | | | | |
| Escherichia coli | 86.1 | 84.8 | 87.8 | 87.4 |
| (MG1655) | | | | |
| Escherichia coli | 86.2 | 84.8 | 87.7 | 87.2 |
| (O157:H7) | | | | |
| Haemophilius influenzae | — | 84.4 | 87.0 | 86.9 |
| Helicobacter pylori | 86.2 | 86.2, 83.2 | 82.6 | 86.2 |
| Klebsiella pneumoniae | 86.4 | 85.8 | 87.4 | 88.5 |
| Lactobacillus delbrueckii | 86.8 | 86.6 | 82.3 | 84.7 |
| Legionella pneumophila | 86.0 | 86.2 | 82.2 | 85.6 |
| Listeria monocytogenes | 86.0 | 86.0 | 83.4 | 87.7 |
| Mycobacterium sp. | — | 86.4, 89.4 | 87.3 | 88.6 |
| Neisseria gonorrhoeae | 84.7 | 85.0 | 86.7 | 87.9 |
| Pantoea ananatis | 86.5 | 86.2 | 88.4 | 87.3 |
| Pseudomonas aeruginosa | 84.3 | 85.6 | 86.9 | 86.7 |
| Rhizobium radiobacter | 85.1 | 87.0 | 84.7 | 85.9 |
| Salmonella choleraesuis | 86.1 | 84.6 | 87.4 | 88.3 |
| Shigella flexneri | 86.2 | 84.8 | 87.7 | 87.3 |
| Spiroplasma citri | 85.7 | 84.8 | 86.1 | 85.6 |
| Staphylococcus aureus | 85.5 | 83.6 | 82.1, 85.1 | 86.9 |
| Staphylococcus | 85.9 | 83.4 | 82.3 | 86.9 |
| epidermidis | | | | |
| Streptoccous pneumoniae | 86.6 | 85.6 | 85.9 | 85.5 |
| Streptococcus pyogenes | 85.8 | 86.4, 84.8 | 85.9 | 85.9 |
| Vibrio cholerae | 85.6 | 85.8 | 87.1 | 86.3 |
| Yersinia enterocolytica | 85.8 | 86.8 | 86.5 | 87.4 |
| HS | — | — | — | — |
| Human K562 | — | — | — | — |
| Water | — | — | — | — |

Additional universal loci are shown in Table 3.

TABLE 4

| | Additional Universal Loci | | | |
|---|---|---|---|---|
| | | | 23S- | |
| ID | 23S-21R | 23S-21F | 24F | 23S-24R |
| Actinomyces naeslundi | 87.8 | 80.8 | 84.8 | — |
| Bacillus cereus | 81.6 | 80.0 | 85.6 | 83.0 |
| Bacillus globigii | 82.0 | 80.0 | 86.2 | 88.0 |
| Bacillus subtilis | 82.0 | 80.2 | 86.2 | 88.0 |
| Bacillus thuringiensis | 81.8 | 80.2 | 85.6 | 83.0 |
| Bacteroides fragilis | 79.2 | 80.0 | 83.6 | 86.0 |
| Bacteroides vulgatus | 79.2 | 79.8 | 83.8 | 87.2 |
| Bordetella pertussis | 83.6 | 80.0 | 85.6 | 83.4 |
| Borrelia burgdorferi | 81.4 | 79.8 | 84.6 | 84.4 |
| Campylobacter jejuni | 81.0 | 79.6 | 82.6 | 83.2 |
| Clostridium difficile | 82.0 | 80.4 | 85.6 | 85.4 |
| Desulfovibrio vulgaris | 84.6 | 79.8 | 86.2 | 84.0 |
| Enterobacter cloacae | 84.2 | 79.0 | 85.8 | 83.4 |
| Enterococcus faecalis | 81.8 | 80.0 | 85.6 | 82.6 |
| Escherichia coli (EDL933) | 84.2 | 79.2 | 86.0 | 83.4 |
| Escherichia coli (MG1655) | 84.2 | 79.2 | 86.2 | 83.4 |
| Escherichia coli (O157:H7) | 84.4 | 79.2 | 86.2 | 83.6 |
| Haemophilius influenzae | 79.4 | 79.2 | 86.0 | 85.4 |
| Helicobacter pylori | 84.6 | 80.2 | 82.6 | 86.4 |
| Klebsiella pneumoniae | 84.0 | 79.2 | 86.2 | 83.4 |
| Lactobacillus delbrueckii | 85.0 | 80.2 | 85.0 | 83.8 |
| Legionella pneumophila | 83.6 | 80.2 | 87.6 | 86.4, 83.4 |
| Listeria monocytogenes | 85.0 | 80.4 | 86.2 | 83.8 |
| Mycobacterium sp. | 86.4 | 81.0 | 87.2 | 83.2 |
| Neisseria gonorrhoeae | 86.0 | 78.8 | 86.0 | 86.8 |
| Pantoea ananatis | 84.4 | 79.2 | 86.2 | 83.6 |
| Pseudomonas aeruginosa | 80.0 | 80.2 | 86.4 | 83.6 |
| Rhizobium radiobacter | 83.6 | 79.8 | 85.8 | 83.2 |
| Salmonella choleraesuis | 84.2 | 79.0 | 86.0 | 83.6 |
| Shigella flexneri | 84.2 | 79.2 | 86.2 | 83.4 |
| Spiroplasma citri | 80.0 | 81.4 | 85.6 | 86.0, 83.6 |
| Staphylococcus aureus | 82.2 | 79.6 | 85.8 | 83.0 |
| Staphylococcus epidermidis | 82.2 | 79.4 | 86.0 | 83.0 |
| Streptoccous pneumoniae | — | 79.2 | 85.6 | 85.2 |
| Streptococcus pyogenes | 83.6 | 79.2 | 85.6 | 85.4 |
| Vibrio cholerae | 79.0 | 79.0 | 85.8 | 83.4 |
| Yersinia enterocolytica | 83.8 | 79.0 | 85.8 | 83.0 |
| HS | — | — | — | — |
| Human K562 | — | — | — | — |
| Water | — | — | — | — |

### Example 3

Coagulase-negative staphylococci are often found among the normal flora of human skin and mucous membrane and have long been regarded as harmless skin commensals and dismissed as culture contaminants. However, both the increasing incidence of coagulase-negative staphylococci as well as a relatively recent recognition that they may play an important role as pathogens has led to the desire to distinguish clinically significant, pathogenic strains from contaminant strains. Using the methods of the present invention, Table 4 represents the differentiation at the species level in a clinical setting for different species of Staphylococcus wherein two melt peaks represent divergence between 16S-23S operon copies.

TABLE 5

| Species | Locus 1 | Locus 2 | Locus 3 | Locus 4 |
|---|---|---|---|---|
| aureus | 85.9 | 83.7 | 82.0, 85.4 | 87.0 |
| capitis | 86.2 | 83.0 | 82.2 | 87.0 |
| epidermidis | 86.2 | 83.3 | 82.2 | 87.0 |
| haemolyticus | 86.0 | 82.2 | 82.2 | 87.0 |
| intermedius | 85.8 | 83.6 | 82.0 | 85.8 |
| saprophyticus | 86.0 | 83.6 | 82.0, 86.0 | 87.0 |
| sciuri | 85.8 | 85.2 | 82.0, 86.0 | 87.4 |
| simulans | 86.4 | 83.8 | 82.1 | 87.2 |
| warnerii | 84.8 | 83.0 | 82.4 | 87.0 |
| xylosus | 86.0 | 83.4 | 82.0, 86.0 | 87.1 |

### Example 3

In this example, reproducibility among strains of S. aureus was tested on a total of 65 independent S. aureus isolates obtained from ATCC (20 isolates) and clinical samples (45 isolates). Average melt temperatures for each locus were within the method-specified acceptance criteria (±0.2° C.). Results obtained with Primer 3 reflect multiple divergent copies of the rRNA operon in S. aureus. The results shown in Table 5 correspond to the genus and species identifications rendered by the providers using standard microbiological approaches.

TABLE 6

| | | | Primer | | Primer |
|---|---|---|---|---|---|
| ID | # Strains | Primer 1 | 2 | Primer 3 | 4 |
| S. aureus | 65 (ATCC + TH) | 85.7 | 83.6 | 82.1, 85.4 | 87.0 |

| | | | | |
|---|---|---|---|---|
| Standard Deviation: | 0.1 | 0.1 | 0.1, 0.1 | 0.2 |

Having described the invention in detail, those skilled in the art will appreciate that modifications may be made of the invention without departing from the spirit and scope thereof. Therefore, it is not intended that the scope of the invention be limited to the specific embodiments described. Rather, it is intended that the appended claims and their equivalents determine the scope of the invention.

1. A method for identifying a sample organism, comprising the steps of:
obtaining a sample of DNA;
isolating a portion of said sample to be analyzed;
cutting said portion into at least two fragments;
ligating both ends of said fragments with double-stranded linker sequences;
adding said fragments to polymerase chain reaction using a dye; and
analyzing said fragments using a melt-curve analysis.

2. The method of claim 1 wherein said sample organism is selected from a group consisting of bacterial, fungal, viral, plant, animal, and combinations thereof.

3. The method of claim 1 wherein said dye is selected from the group consisting of SYBR green-intercalating dyes, cyanine dimer dyes, ethidium bromide dyes, and acridine dyes.

4. The method of claim 1 wherein said cutting step further comprises enzymatically cutting said sample by using a restriction endonuclease that recognizes a specific DNA cut site.

5. The method of claim 4 wherein said isolation step further comprises treating said fragment with a phosphatase that removes phosphates from each end of said fragment.

6. The method of claim 1 wherein said adding step further comprises using at least one said fragment as a template.

7. The method of claim 4 further comprising the step of:
amplifying said fragments using one primer complementary to said ligated linker sequence and one primer complementary to a universal DNA sequence, wherein amplification produces at least two new strands.

8. The method of claim 1 wherein said analyzing step further comprises:
denaturing said fragments at a high temperature;
lowering said temperature until said fragments reassemble;
depositing said dye between said fragments as said fragments are reassembling;
increasing said temperature until said fragments begin to break apart;
monitoring said dye and said fragments as said temperature is increased;
recording at least three data points; and
analyzing said data points with known information to identify said sample.

9. The method of claim 1 wherein said sample has a length of from about 0.1 nanograms to 1000 nanograms.

10. The method of claim 1 wherein said method is automated.

11. A method for identifying a sample organism, comprising the steps of:
obtaining a sample of DNA;
cutting said sample by using a restriction endonuclease that recognizes a specific DNA cut site, wherein cutting produces at least one fragment of said sample;
treating said cut sample with a phosphatase to remove phosphates from ends of said fragments;
ligating a double-stranded linker sequence to each end of said fragments;
adding at least one said fragment to a polymerase chain reaction using a SYBR green-intercalating dye, wherein said fragment is used as a template;
amplifying said fragments by using one primer complementary to said ligated linker sequence and one primer complementary to a universal DNA sequence, wherein amplification produces at least two strands;
denaturing at least two said strands at a high temperature;
lowering said temperature until said strands reassemble, wherein said SYBR green dye is deposited between said strands;
recording at least three data points as said temperature is slowly increased;
comparing said data points with known information about organisms; and
determining said identification of said sample organism.
